(19) United States
(12) Patent Application Publication
(10) Pub. No.: US 2014/0162968 A1
(43) Pub. Date: Jun. 12, 2014

(54) METHOD FOR SCREENING OF AGENT FOR TREATING OR PREVENTING OBESITY

(71) Applicant: Myeong Ok Kim, Gyeongsangnam-do (KR)

(72) Inventor: Myeong Ok Kim, Gyeongsangnam-do (KR)

(21) Appl. No.: 14/232,605

(22) PCT Filed: Jan. 20, 2012
(86) PCT No.: PCT/KR2012/000579

§ 371 Date: Feb. 27, 2014

(30) Foreign Application Priority Data
Jul. 14, 2011 (KR) 10-2011-0070125

(51) Int. Cl. G01N 33/68; A61K 31/7048

(52) U.S. Cl. CPC G01N 33/6872; A61K 31/7048
USPC 514/27; 435/6.13

(57) ABSTRACT

The present invention relates to a method for screening and preparing an obesity prevention or treatment agent, and a method for preparing an obesity prevention or treatment agent. Being able to easily detect a substance that has a preventive and therapeutic effect on obesity, the present invention has a wide spectrum of applications in the research and medicine field in the prevention or treatment of obesity.

### BACKGROUND OF THE INVENTION

1. Field of the Invention

The present invention relates to a method for screening an obesity prevention or treatment agent, and a method for preparing an obesity prevention or treatment agent.

2. Description of the Related Art

Obesity is a medical condition in which excess body fat has accumulated to the extent that it may have an adverse effect on health. This does not refer to a condition of excessive bodyweight, but rather a condition where fat has excessively accumulated in the body due to a metabolic disorder. That is, calorie intake exceeds energy required for body activity and growth so that calories are excessively accumulated in the form of triglycerides in adipose tissues, thus causing obesity. Even in itself, obesity not only prompts disfigurement, discomfort and disability, but also causes various diseases, including cardiovascular diseases, such as hyperlipidemia, hypercholesterolemia, hypertension, arteriosclerosis, myocardial infarction and the like, as well as renal disease, type II insulin-independent diabetes, and pulmonary disease, which may lead to death. In advanced countries, 30% of the adult population is obese, and particularly, obese men in a young age group (25-34 years old) were measured to have about a 12-times higher mortality rate than normal men, predominantly due to cardiovascular diseases.

In order to treat obesity, studies are currently being performed on diet therapy, exercise therapy, behavior modification therapy, surgical therapy, drug therapy and the like. Of them, drugs for treating obesity have been particularly extensively developed, as exemplified by fat accumulation inhibitors (appetite inhibitors, and agents for suppressing food absorption or fatty acid production) and fat utilization stimulants (thermogenic or lipolytic agents). Fluoxetine, orlistat, and sibutramine have recently been widely used in the clinical field. However, fluoxetine (sold under the trade name “Prozac”), used as an antidepressant with the function of selectively inhibiting serotonin reuptake, is known to have only a temporary effect on the reduction of bodyweight, but with the concomitant occurrence of side effects, such as enervation, perspiration and lethargy. Orlistat (sold under the trade name “Xenical”) inhibits the activity of lipase in the small intestines to reduce fat absorption by about 30%, but causes steatorrhea and requires the supplement of fat-soluble vitamins with long-term administration. Sibutramine (sold under the trade name “Reductil”) shows the double action of inhibiting the reuptake of serotonin and norepinephrine. An increase in serotonin activates the sympathetic system to incite an exothermic reaction in brown fat tissue, but causes side effects, such as blood pressure increase, mouth drying, constipation and insomnia. Therefore, there is a pressing need for the development of an anti-obesity agent that is safe and effective.

Recent studies have demonstrated that the expression of neuropeptide Y (NPY), which mediates the action of reptin in the hypothalamus, is upregulated in reptin/reptin receptor-deficient mice, and that the deletion of the neuropeptide Y gene is less likely to make reptin-deficient mice obese. Neuropeptide Y is a 36-amino acid neuropeptide secreted from the pancreas which belongs to the neuroendocrine peptide family, and is abundantly found in the mammalian central and peripheral nervous systems, particularly, in the hypothalamus and the cortex.

Meanwhile, GABA (Gamma Amino Butyric Acid), a kind of non-protein amino acids widely found in nature, is the chief inhibitory neurotransmitter in the mammalian brain and spinal cords. It plays a role in various physiological mechanisms, including the activation of the metabolism of cerebral cells by increasing cerebral blood flow and oxygen supply. GABA is found at high concentrations in the cerebral cortex and the cerebellar gray and white matter, and distributed over almost all areas of the brain, such as the hippocampus, thalamus, striatum, olfactory bulb, myelencephalon, etc. When released to synapses, GABA binds to membrane proteins of postsynaptic neurons via GABA receptors. There are two classes of GABA receptors: GABA_{A} and GABA_{B}. GABA_{A} receptors are ligand-gated ion channels which are opened upon activation to allow the selective influx of Cl⁻ through their pores, resulting in hyperpolarization of the neuron whereas GABA_{B} receptors are G protein-coupled receptors which can stimulate the opening of K⁺ channels, showing indirect inhibitory activity. However, thus far there has been no definite correlation suggested between GABA_{B} receptors and obesity nor between GABA_{B} receptors and neuropeptide Y, nor for the mechanisms of GABA_{B} receptors and neuropeptide Y for the prevention and treatment of obesity.

In the background of this pressing need for safe anti-obesity agents that do not provoke side effects, intensive and thorough research into the screening of such anti-obesity agents resulted in the finding that candidate substances for the effective and safe therapy of obesity can be screened by monitoring changes in the expression level of GABA_{B} receptors and neuropeptide Y, which led to the present invention.

### SUMMARY OF THE INVENTION

It is an object of the present invention to provide a method for screening an obesity prevention or treatment agent, comprising: (a) measuring expression level of GABA_{B} receptor and neuropeptide Y in a subject; (b) administering a candidate substance to the subject, followed by measuring expression level of GABA_{B} receptor and neuropeptide Y in the subject; and (c) determining the candidate substance as an obesity prevention or treatment agent when the expression level of GABA_{B} receptor in step (b) is found to have increased compared to step (a) and the expression level of neuropeptide Y in step (b) is found to have decreased compared to step (a).

It is another object of the present invention to provide a method for preparing an obesity prevention or treatment agent, comprising (a) administering a candidate substance to a subject, followed by examining upregulated expression of GABA_{B} receptor and downregulated expression of neuropeptide Y in the subject; and (b) adding the candidate substance to a composition when the candidate substance upregulates the expression of GABA_{B} receptor and downregulates the expression of neuropeptide.

### Effect of the Invention

Being able to easily detect a substance that has a preventive and therapeutic effect on obesity, the present invention has a wide spectrum of applications in the research and medicine fields for the prevention or treatment of obesity.

### BRIEF DESCRIPTION OF THE DRAWINGS

FIG. 1 shows graphs of body weight changes (A) and daily food uptake (B) in animals with or without administration of anthocyanin.

FIG. 2 shows fluorescent images comparing the sizes of epididymal white adipose tissues excised from animals without administration of anthocyanin (A) or with administration of anthocyanin at a dose of 6 mg (B) and 24 mg (C).

FIG. 3 shows an image of GABA_{B} receptor blots illustrating expression level in the hypothalamus of animals with or without administration of anthocyanin, as measured by Western blotting (A), and a graph analyzing densities of the protein according to the group (B).

FIG. 4 shows an image of neuropeptide Y blots illustrating expression level in the hypothalamus of animals with or without administration of anthocyanin, as measured by Western blotting (A), and a graph analyzing densities of the protein according to the group (B).

FIG. 5 shows images of PKA-α (A) and p-CREB (B) blots illustrating expression levels in the hypothalamus of animals with or without administration of anthocyanin, as measured by Western blotting, and graphs analyzing densities of the proteins according to the group.

### DETAILED DESCRIPTION OF THE PREFERRED EMBODIMENTS

In accordance with an aspect thereof, the present invention provides a method for screening an obesity preventive or therapeutic agent, comprising administering a candidate substance to a subject and measuring expression levels of GABA_{B} receptor and neuropeptide Y. In detail, the screening method of the present invention comprises: (a) measuring expression level of GABA_{B} receptor and neuropeptide Y in a subject; (b) administering a candidate substance to the subject, followed by measuring expression level of GABA_{B} receptor and neuropeptide Y in the subject; and (c) determining the candidate substance as an obesity prevention or treatment agent when the expression level of GABA_{B} receptor in step (b) is found to have increased compared to step (a) and the expression level of neuropeptide Y in step (b) is found to have decreased compared to step (a).

In accordance with another aspect thereof, the present invention provides a method for preparing an obesity prevention or treatment agent, comprising (a) administering a candidate substance to a subject, followed by examining upregulated expression of GABA_{B} receptor and downregulated expression of neuropeptide Y in the subject; and (b) adding the candidate substance to a composition when the candidate substance upregulates the expression of GABA_{B} receptor and downregulates the expression of neuropeptide.

As used herein, the term “subject” refers to any mammal that expresses GABA_{B} receptor and neutopeptide Y, including, but not limited to, dogs, cows, horses, rabbits, mice, rats, chickens, and humans. Preferably, rats may be employed to measure expression levels of GABA_{B} receptor and neuropeptide Y.

As used herein, “GABA_{B} receptor” is a receptor that responds to the neurotransmitter GABA (Gamma Amino Butyric Acid), and is composed of two subunits GABA_{B1} and GABA_{B2}. The GABA_{B} receptor is a member of the superfamily of G protein-coupled receptors, also known as seven-transmembrane domain receptors. When activated, the GABA_{B} receptor stimulates the opening of K⁺ channels to exhibit indirect inhibition, modulating the secretion of inhibitory neurotransmitters. Correlation between GABA_{B} receptor and obesity has not yet been definitely described until now. The present inventors first found the upregulated expression of GABA_{B} receptor by an anti-obesity substance (anthocyanin), which has been developed to a screening method by which a candidate substance is determined as an obesity prevention and treatment agent if the expression of GABA_{B} receptor is upregulated by treatment with the candidate substance.

The term “neuropeptide Y (NPY),” as used herein, means a 36-amino acid neuropeptide belonging to the neuroendocrine peptide family which is abundantly found in the mammalian central and peripheral nervous systems, particularly, in the hypothalamus and the cortex. However, thus far there have been no definite suggestions for a correlation between GABA_{B} receptor and obesity nor between GABA_{B} receptor and neuropeptide Y, nor for mechanisms of GABA_{B} receptor and neuropeptide Y on the prevention and treatment of obesity. Based on their first discovery that an anti-obesity substance (anthocyanin) upregulates the expression of GABA_{B} receptor and down-regulates the expression of neuropeptide Y, the present inventors designed a screening method by which a candidate substance is determined as an obesity prevention and treatment agent if the candidate substance acts to upregulate the expression of GABA_{B} receptor and downregulate downregulate the expression of neuropeptide Y. It was first found by the present inventors that an anti-obesity substance decreases the expression level of neuropeptide Y and increases the expression level of GABA_{B} receptor.

As used herein, the term “candidate substance” means any substance that is expected to prevent or treat obesity, and particularly refers to a target to be tested for ability to prevent and treat obesity through the mechanism of modulating expression of GABA_{B} receptor and neuropeptide Y. Examples of the candidate substance include, but are not limited to, proteins, oligopeptides, small organic molecules, polysaccharides, polynucleotides and various compounds. They may be synthetic as well as natural.

As used herein, the term “obesity prevention agent” is intended to refer to any substance that brings about the suppression or delay of the onset of certain obesity-induced diseases thanks to the administration thereof. The term “obesity treatment agent” is intended to refer to any substance that brings about improvements in symptoms of certain obesity-induced diseases or the beneficial alteration of the diseases thanks to the administration thereof.

The composition to which the candidate substance is added in the preparation method of an obesity prevention or treatment agent may further comprise a pharmaceutically acceptable vehicle. The composition comprising a pharmaceutically acceptable vehicle may be in various oral or non-oral dosage forms. In this regard, the composition of the present invention may be formulated in combination with a diluent or excipient such as a filler, a thickener, a binder, a wetting agent, a disintegrant, a surfactant, etc. Solid preparations intended for oral administration may be in the form of tablets, pills, powders, granules, capsules, and the like. In regards to these solid agents, the active ingredient of the present invention is formulated in combination with at least one excipient such as starch, calcium carbonate, sucrose, lactose, or gelatin. In addition to a simple excipient, a lubricant such as magnesium stearate, talc, etc. may be used. Among liquid preparations intended for oral administration are suspensions, internal use solutions, emulsion, syrups, and the like. Plus a simple diluent such as water or liquid paraffin, various excipients, such as wetting agents, sweeteners, aromatics, preservatives, and the like may be contained in the liquid preparations. Also, the composition may be in a parenteral dosage form such as sterile aqueous solutions, non-aqueous solvents, suspensions, emulsions, lyophilizates, suppositories, and the like. Injectable propylene glycol, polyethylene glycol, vegetable oils such as olive oil, and esters such as ethyl oleate may be suitable for the non-aqueous solvents and suspensions. The basic materials of suppositories include Witepsol, macrogol, Tween 61, cacao butter, laurin butter, and glycerogelatin. The composition may be in a dosage form selected from the group consisting of a tablet, a pill, a powder, a granule, a capsule, a suspension, an internal use solution, an emulsion, a syrup, an sterile aqueous solution, a non-aqueous solvent, a suspension, a lyophilizate, and a suppository.

The term “obesity,” as used herein, means a condition where fat is excessively accumulated in the body due to a metabolic disorder. Obesity is known to prompt disfigurement, discomfort and disability in itself, but also causes various diseases, including cardiovascular diseases, such as hyperlipidemia, hypercholesterolemia, hypertension, arteriosclerosis, myocardial infarction and the like, renal disease, type II insulin-independent diabetes, and pulmonary disease.

The term “screening,” as used herein, means finding out a certain property, including susceptibility to or activity on a certain compound, such as an antibiotic, an enzyme, etc.

In the present invention, after administration with a candidate substance which is expected to upregulate the expression of GABA_{B} receptor and to downregulate the expression of neuropeptide Y, rats which express GABA_{B} receptor and neuropeptide Y are monitored for expression patterns of GABA_{B} receptor and neuropeptide Y to examine whether the candidate substance can be used as an obesity treatment agent by modulating the expression of the proteins. In detail, when a candidate substance is observed to upregulate the expression of GABA_{B} receptor while down-regulating the expression of neuropeptide Y in the subject administered therewith, the candidate can be determined to be an obesity prevention and treatment agent. For the screening of obesity prevention or treatment agents, preferably, reference may be made to expression levels of GABA_{B} receptor and neuropeptide Y in the hypothalamus.

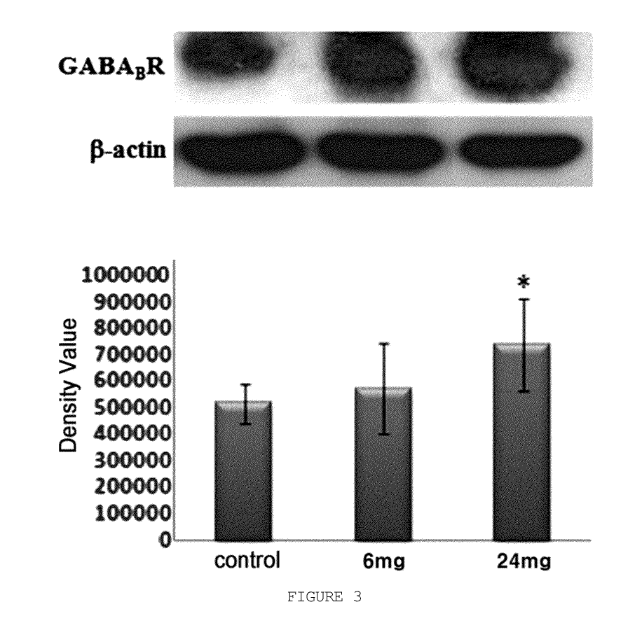

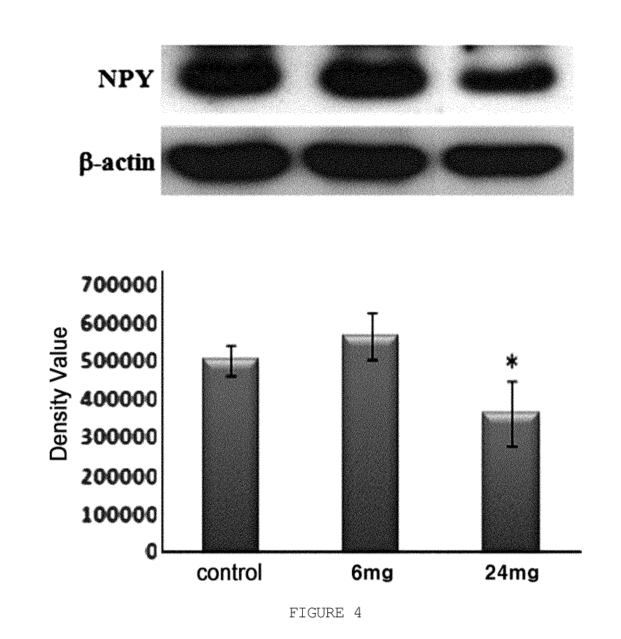

In one embodiment of the present invention, anthocyanin, known to exert an anti-obesity effect on adult rats, was demonstrated to induce the upregulated expression of GABA_{B} receptor (FIG. 3) and the downregulated expression of neuropeptide Y (FIG. 4) in rats, indicating that a modulation in the expression level of GABA_{B} receptor and neuropeptides Y can be used as an index for screening an obesity prevention and treatment agent.

In one embodiment, the screening method according to the present invention further comprising measuring expression level of PKA-α or p-CREB in the steps (a) and (b), and determining the candidate substance as an obesity prevention or treatment agent when the expression level of PKA-α in step (b) is found to have decreased compared to step (a) or the expression level of p-CREB in step (b) is found to have increased compared to step (a).

In the screening method of the present invention, the candidate substance may be confirmed as usable as an obesity prevention and treatment agent or not by monitoring a modulation in the expression levels of PKA-α and p-CREB, the activities of both of which are controlled with regard to the neurotransmission of GABA_{B} receptor. In detail, if a candidate substance that acts to upregulate the expression of GABA_{B} receptor and downregulate the expression of neuropeptide Y in a subject is further observed to decrease the expression of PKA-α and increase the expression of p-CREB, simultaneously, in the subject, the candidate substance is confirmatively determined as an obesity prevention and treatment agent.

As used herein, the term “PKA-α (protein kinase A-α)” refers to a protein acting as a second messenger in GABA receptors, which is reported to induce various intracellular changes including cAMP, activate protein kinases through a change in the phosphorylation thereof, and to control the expression levels of downstream genes.

As used herein, the term “CREB (cyclic AMP response element binding protein)” is a cellular transcription factor which is phosphorylated by activated PKA to increase the description of related genes. Particularly, CREB is known as an essential transcription factor which continues to be expressed in preadipocytes before and during differentiation to adipocytes and binds to promoters of adipocyte-specific genes to regulate the description of the genes.

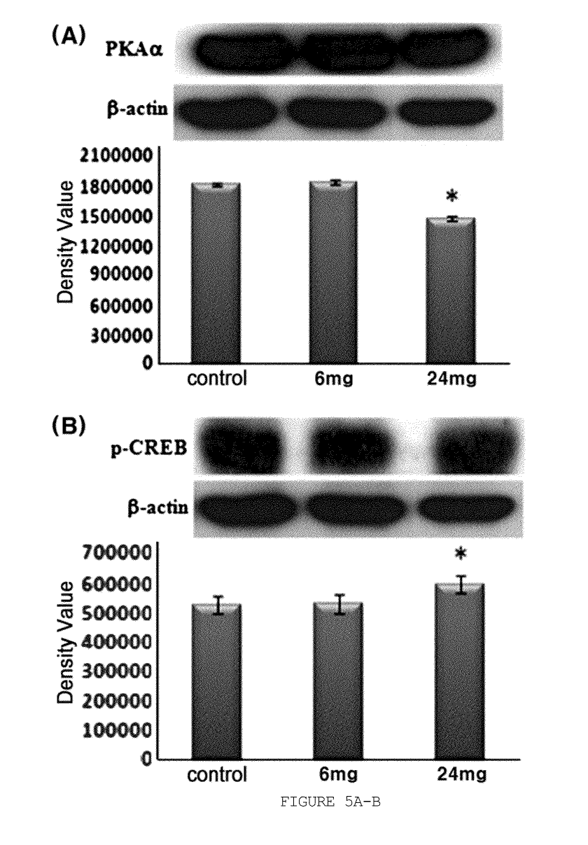

In one embodiment, anthocyanin, known to exert an anti-obesity effect on rats, was demonstrated to induce the downregulated expression of PKA-α receptor (FIG. 5A) and the upregulated expression of p-CREB (FIG. 5B) in rats, indicating that a modulation in the expression levels of PKA-α and p-CREB can be used as an additional index for screening an obesity prevention and treatment agent.

In the screening method of the present invention, the measurement of expression modulations of GABA_{B} receptor, neuropeptide Y, PKA-α, and p-CREB may be achieved using any technique that is typically used in the art. The expression of GABA_{B} receptor, neuropeptide Y, PKA-α, and p-CREB may be quantitatively analyzed at the level of mRNAs or proteins encoded by the mRNAs. For the quantitation of mRNAs, for example, complementary primer or probe sequences may be used while protein levels may be determined using antibody binding to proteins or their fragments. Preferably, the expression of GABA_{B} receptor, neuropeptide Y, PKA-α or p-CREB may be measured at a protein level using Western blot.

As used herein, the term “determination of mRNA expression level” refers to a process of measuring the mRNA level of GABA_{B} receptor, neuropeptide Y, PKA-α, or p-CREB in a subject administered with a candidate substance to screen an obesity prevention and treatment agent. To this end, the technique of measuring mRNA levels may include, but is not limited to, reverse transcription polymerase chain reaction (RT-PCR), competitive RT-PCR, real-time RT-PCR, RPA (RNase protection assay), Northern blotting, and DNA chip.

The “determination of protein expression level” in the present invention is a process of qualitatively and quantitatively analyzing proteins expressed from the mRNAs of GABA_{B} receptor, neuropeptide Y, PKA-α or p-CREB in a subject administered with a candidate substance to screen an obesity prevention and treatment agent, preferably using antibodies specifically binding to the proteins of the genes. In this regard, the measurement of the protein levels may be achieved by an immunoassay using an antibody specific for each of GABA_{B} receptor, neuropeptide Y, PKA-α and p-CREB, examples of which include, but are not limited to, Western blotting, ELISA (enzyme linked immunosorbent assay), radioimmunoassay (RIA), radioimmunodiffusion, ouchterlony immunodiffusion, rocket immunoelectrophoresis, histoimmunochemical staining, immunoprecipitation assay, complement fixation assay, FACS, and protein chip. Further, analysis using an antibody may recruit a detectable marker-labeled second antibody specific for a target protein while a detectable marker-labeled third antibody having affinity for the second antibody may be optionally used. The detectable marker labeled to the second or the third antibody may be an enzyme which can develop a color while being cultured in the presence of a suitable chromogenic substrate. The detectable maker can include compositions that are detectable by spectroscopicc, enzymatic, photochemical, biochemical, bioelectronic, immunochemical, electric, optical, or chemical means, as exemplified by, but not limited to, fluorescent markers and dyes, magnetic labels, linked enzymes, mass spectrometry tags, spin labels, electron transfer donors and acceptors, and the like.

A better understanding of the present invention may be obtained through the following examples which are set forth to illustrate, but are not to be construed as limiting the present invention.

### Reference Example 1

### Preparation of Tissues and Samples

For hematoxylin-eosin staining, an epididymal white adipose tissue (WAT) was fixed at 4° C. for 72 hrs in ice-chilled 1× PBS containing 4% para-formaldehyde, and immersed at 4° C. for 72 hrs in 20% sucrose sucrose phosphate buffer for cryoprotection. The fixed tissue was frozen with O.C.T compound (A. O. USA), and 10 μm coronal sections were made in coronal planes (Leica cryostat CM 3050, Germany). The sections were mounted on a probe-on, positively charged slide at room temperature and stored at −70° C. until use.

### Example 1

### Assay for Preventive and Therapeutic Effect of Anthocyanin on Obesity

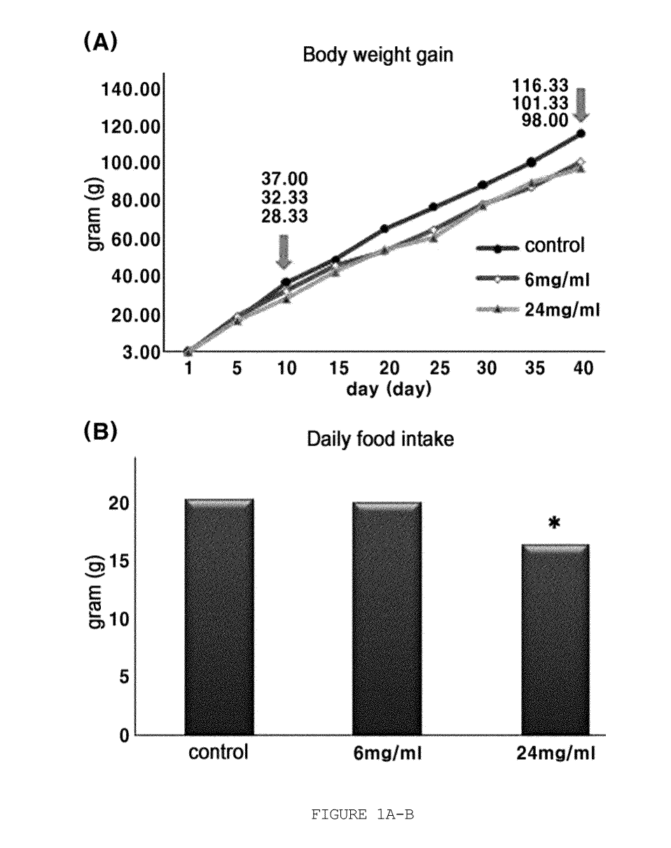

For use in assay for the preventive and therapeutic effect of anthocyanin on obesity, male Sprague-Dawley rats, 60 days old (the Experimental Animal Breeding Center of the Gyeongsang National University, Jinju, Korea)) were divided groups of four which were then orally administered with anthocyanin at a dose of 6 mg and 24 mg, respectively, or not administered with anthocyanin for a control. They were maintained for 40 days in a temperature-controlled condition with lighting 06:00-20:00. While being maintained, the rats in each group were monitored for weight change and daily food uptake, and the results are shown in FIG. 1. FIG. 1 shows graphs of weight changes (A) and daily food uptake (B) in animals with or without administration of anthocyanin.

As can be seen in FIG. 1, weight was increased by 69 g from 32.33 g to 101.33 g in the group administered with 6 mg of anthocyanin and by 69.67 g from 28.33 g to 98 g in the group administered with 24 mg of anthocyanin. The anthocyanin-administered rats were significantly less apt to gain weight, compared to the non-administered control which was measured to increase in weight by 79.33 g from 37 g to 116.33 g (FIG. 1A). In addition, daily food uptake was significantly decreased in the group administered with 24 mg of anthocyanin (16.43 g), compared to the control (20.03 g) (FIG. 1B).

### Example 2

### Measurement of Epididymal White Adipose Tissue

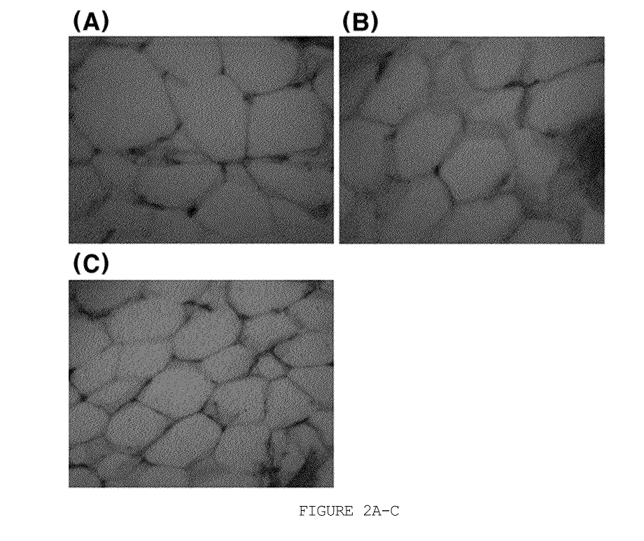

The animals bred as in Example 1 were collectively sacrificed at 10:00 AM 40 days after the oral administration, and epididymal white adipose tissue (WAT) was excised from each sacrificed animal and used for comparison of obesity. The epididymal WAT was fixed at 4° C. for 72 hrs in ice-chilled 1× PBS containing 4% para-formaldehyde, and immersed at 4° C. for 72 hrs in a 20% sucrose phosphate buffer for cryoprotection. The fixed tissue was frozen with O.C.T compound (A. O. USA), and 10 μm coronal sections were made in coronal planes (Leica cryostat CM 3050, Germany). The sections were mounted on probe-on, positively charged slides and thawed at room temperature for 3 hrs. The thawed slides were washed twice for 15 min in PBS, stained for 3 min with hematoxylin-eosin, and then washed again for 1 min with distilled water, followed by treatment with ethanol (70 to 100%) and 100% xylene for 3 min each. Cover slips were mounted on the slides which were observed under a fluorescence microscope at 400× magnification. The results are given in FIG. 2. FIG. 2 shows fluorescent images comparing sizes of epididymal white adipose tissues excised from animals without administration of anthocyanin (A) or with anthocyanin at a dose of 6 mg (B) and 24 mg (C).

As can be seen in FIG. 2, the number of adipocytes per unit area was larger in the animals administered with anthocyanin at a dose of 6 mg (22.47 cells/250 mm²) (B) and 24 mg (22.8 cells/250 mm²) (C) than in the control (18.75 cells/250 mm²) (A), indicating adipocytes were reduced in size by treatment with anthocyanin. These data suggested that anthocyanin can be used as an obesity prevention and treatment agent.

### Example 3

### Effect of Obesity Prevention and Treatment Agent on Expression of GABA_{B} Receptor

To examine the effect of an obesity prevention and treatment agent on the expression of GABA_{B} receptor, the animals maintained as in Example 1 were collectively sacrificed at 10:00 AM 40 days after the oral administration, and the hypothalamus was excised from each of them. The hypothalamic sample was homogenized in 0.2 M PBS containing a protease inhibitor cocktail. Protein levels were measured using a Bio-Rad protein analysis solution, and after two rounds of ultracentrifugation at 4° C. and 12,000 rpm for 20 min, the supernatants containing proteins were separated. The supernatants were loaded in an aliquot of 30 μl per lane to 10-18% gel and subjected to SDS-PAGE, followed by transfer to a polyvinylidene difluoride (PVDF) membrane. This membrane was blocked with 5% (v/v) skim milk to reduce non-specific binding, and incubated with a primary goat anti-GABA_{B}R1 antibody (1:200; Santa Cruz Biotechnology, Santa Cruz, Calif., USA) and an anti-beta-actin antibody for quantitative comparison, followed by immune reaction with a secondary HRP (horseradish peroxidase)-conjugated anti-goat and anti-rabbit IgG (1:1000; Santa Cruz Biotechnology, Santa Cruz, Calif., USA). Protein levels were determined by chemiluminescence using ECL-detecting reagent (Amersham Pharmacia Biotech, Western blotting detection reagents). Western blots on the X-ray film were analyzed by densitometry using computer-based Sigma Gel (SPSS Inc. Chicago, USA), and the results are shown in FIG. 3. FIG. 3 shows an image of GABA_{B} receptor blots illustrating expression levels in the hypothalamus of animals with or without administration of anthocyanin, as measured by Western blotting (A), and a graph analyzing densities of the protein according to the group (B).

As can be seen in FIG. 3, the animals which were prevented from becoming obese by being administered anthocyanin were observed to have an increased expression level of GABA_{B} receptor in the hypothalamus, suggesting that the upregulated expression of GABA_{B} receptor in the hypothalamus can be used as an index for screening an obesity prevention and treatment agent.

### Example 4

### Effect of Obesity Prevention and Treatment Agent on Expression of Neuropeptide Y

To examine the effect of an obesity prevention and treatment agent on the expression of neuropeptide Y, expression level of neuropeptide Y was measured in a manner similar to that of Example 3, except for using a primary goat anti-NPY antibody (1:200; Santa Cruz Biotechnology, Santa Cruz, Calif., USA), and the results are given in FIG. 4. FIG. 4 shows an image of neuropeptide Y blots illustrating expression level in the hypothalamus of animals administered anthocyanin and those not, as measured by Western blotting (A), and a graph analyzing densities of the protein according to the group (B).

As can be seen in FIG. 4, the animals which were prevented from being obese by administration with 24 mg of anthocyanin were observed to have a decreased expression level of neuropeptide Y in the hypothalamus, suggesting that the downregulated expression of neuropeptide Y in the hypothalamus can be used as an index for screening an obesity prevention and treatment agent.

### Example 5

### Effect of Obesity Prevention and Treatment Agent on Expression of Proteins Downstream of GABA_{B} Receptor

An examination was made of the effect of an upregulated expression of GABA_{B} receptor on the downstream signaling mechanism in the hypothalamus of an obesity-suppressed animal. In this regard, PKA-α and p-CREB, both known to modulate in expression depending on GABA_{B} receptor, were analyzed for expression level in the same manner as in Example 3, except for using a primary goat anti-PKAα antibody (1:200; Santa Cruz Biotechnology, Santa Cruz, Calif., USA) and a primary goat anti-p-CREB antibody (1:200; Santa Cruz Biotechnology, Santa Cruz, Calif., USA), and the results are given in FIG. 5. FIG. 5 shows images of PKA-α (A) and p-CREB (B) blots illustrating expression level in the hypothalamus of animals administered anthocyanin and those not, as measured by Western blotting, and graphs analyzing densities of the proteins according to the group.

As can be seen in FIG. 5, the animal group which was prevented from being obese by administration with 24 mg of anthocyanin was observed to show a decrease in the expression level of PKA-α in the hypothalamus (FIG. 5A), but to show an increase in the expression level of p-CREB (FIG. 5B). Taken together, these results demonstrate that modulated expression levels of PKA-α and p-CREB in the hypothalamus of a subject can be used as an additional index for screening an obesity prevention and treatment agent.

What is claimed is:

1. A method for screening an obesity prevention or treatment agent, comprising:
(a) measuring expression level of GABA_{B} receptor and neuropeptide Y in a subject;
(b) administering a candidate substance to the subject, followed by measuring expression level of GABA_{B} receptor and neuropeptide Y in the subject; and
(c) determining the candidate substance as an obesity prevention or treatment agent when the expression level of GABA_{B} receptor in step (b) is found to have increased compared to step (a) and the expression level of neuropeptide Y in step (b) is found to have decreased compared to step (a).

2. The method of claim 1, further comprising measuring expression level of PKA-α or p-CREB in steps (a) and (b), and determining the candidate substance as an obesity prevention or treatment agent when the expression level of PKA-α in step (b) is found to have decreased compared to step (a) or the expression level of p-CREB in step (b) is found to have increased compared to step (a).

3. The method of claim 1, wherein the expression level of GABA_{B} receptor and neuropeptide Y is measured in a hypothalamus.

4. The method of claim 1, wherein the subject is a rat.

5. The method of claim 1, wherein the expression level of GABA_{B} receptor and neuropeptide Y is measured at an mRNA or protein level.

6. A method for preparing an obesity prevention or treatment agent, comprising:
(a) administering a candidate substance to a subject, followed by examining upregulated expression of GABA_{B} receptor and downregulated expression of neuropeptide Y; and
(b) adding the candidate substance to a composition when the candidate substance upregulates the expression of GABA_{B} receptor and downregulates the expression of neuropeptide Y.

7. The method of claim 6, further comprising examining downregulated expression of PKA-α and upregulated expression of p-CREB in step (a), and adding the candidate substance to a composition when the candidate substance down-regulates the expression of PKA-α and upregulates the expression of p-CREB in step (b).

8. The method of claim 6, wherein the upregulated expression of GABA_{B} receptor and the downregulated expression of neuropeptide Y are examined in a hypothalamus.

9. The method of claim 6, wherein the subject is a rat.

10. The method of claim 6, wherein the upregulated expression of GABA_{B} receptor and the downregulated expression of neuropeptide Y are examined at an mRNA or protein level.
